(19) United States
(12) Patent Application Publication
(10) Pub. No.: US 2025/0325750 A1
(43) Pub. Date: Oct. 23, 2025

(54) Multi-Barrel Syringe for Sequential Delivery of Fluids and Methods of Use

(71) Applicant: Becton, Dickinson and Company, Franklin Lakes, NJ (US)

(72) Inventors: Rahul Malviya, Salmon Arm (CA); Amarsinh Deeliprao Jadhav, Hyderabad (IN); Narsi Reddy Sanikommu, Kanigiri (IN)

(21) Appl. No.: 18/868,207

(22) PCT Filed: Jun. 15, 2023
(86) PCT No.: PCT/US23/25355

§ 371 Date: Nov. 22, 2024

(30) Foreign Application Priority Data
Jun. 24, 2022 (IN) 202211036491

(51) Int. Cl. A61M 5/19; A61M 5/315

(52) U.S. Cl. CPC A61M 5/19; A61M 5/31596; A61M 2005/31598

(57) ABSTRACT

A multi-barrel syringe for sequential expulsion of a first fluid followed by a second fluid includes an outer barrel containing the first fluid and an inner barrel slideably inserted in the outer barrel containing the second fluid. The outer barrel includes an open proximal end, a distal end comprising a fluid port, a sidewall extending between the proximal end and the distal end, and a protrusion extending into the outer barrel from an inner surface of the outer barrel. The inner barrel includes an open proximal end, a closed distal end, and a sidewall extending between the proximal end and the distal end. Moving the inner barrel through the outer barrel brings a pierceable portion of the inner barrel into contact with the protrusion of the outer barrel to pierce the inner barrel, thereby allowing the second fluid to flow from the inner barrel to the outer barrel.

### CROSS-REFERENCE TO RELATED APPLICATION

The present application claims priority to Indian Patent Application No. 202211036491 entitled “Multi-Barrel Syringe for Sequential Delivery of Fluids and Methods of Use” filed Jun. 24, 2022, the entire disclosure of which is hereby incorporated by reference in its entirety.

### BACKGROUND OF THE INVENTION

### Field of the Invention

The present disclosure relates generally to syringes for expelling multiple fluids and, in particular, to a syringe for sequential expulsion of a first fluid, such as a medical fluid, followed by a second fluid, such as a flush solution.

### Description of Related Art

Vascular access devices (VADs) are commonly used medical devices, which can include intravenous (IV) catheters, such as peripheral catheters or central venous catheters. If not properly maintained or if exposed to a non-sterile environment, the VADs can become contaminated, sealed with blood clots, and/or can spread infection. Many medical facilities implement sterile practices and protocols to ensure that VADs are used properly and do not become sealed or infected. These protocols often include sterilizing the VAD and flushing the catheter with a flush solution. Specifically, VAD standards of practice usually recommend flush procedures be performed after catheter placement, before fluid infusion, and before and after drug administration, blood sampling, transfusions, and/or administration of parenteral nutrition.

These flush procedures are intended to confirm catheter patency, avoid drug incompatibilities, ensure that the complete drug dose is administered to the patient, prevent thrombus formation, and minimize a risk of bloodstream infections caused by contamination of the VAD. Further, flushing can prevent build-up of deposits of blood, blood residue, and IV drugs within a catheter or other VAD device. Such build-up can cause partial or complete blockage of the fluid pathway in a catheter system, which may require a practitioner to perform certain expensive and potentially dangerous processes for purging the affected catheter or may require the practitioner to exchange the blocked catheter with a new catheter. Often, such blockages lead to interruptions in therapy, which may compromise patient care. The build-up of residue within a catheter may also increase infection risk.

During administration of a drug or another medical fluid, a catheter or VAD is usually flushed both before and after delivery of the fluid from a syringe or from another drug delivery device to the VAD. In some cases, catheters can be flushed using syringe assemblies that are pre-filled with various fluids (referred to herein as flush solutions). The size of the pre-filled syringe used to flush the catheter varies based, for example, on a size and length of the catheter. Typically, pre-filled syringes for flushing a catheter or IV line contain a volume of about 1 mL, 3 mL, 5 mL, or 10 mL of flush solution. Many different syringes commercially available from different manufacturers can be used for providing the flush solution through a catheter or IV line of a patient including, for example, the BD PosiFlush™ pre-filled saline syringe by Becton, Dickinson and Company.

In order to perform a flushing procedure, a practitioner connects the pre-filled flushing syringe to the VAD and advances a plunger rod into a barrel of the pre-filled syringe, which expels the flush solution through a nozzle or fluid port of the pre-filled syringe to the VAD. After flushing, the pre-filled syringe is disconnected from the VAD so that the practitioner can administer a dose of the medical fluid. The medical fluid is conventionally contained in a vial and is drawn into a separate syringe which, after being filled with the medical fluid, is connected to the VAD. The medical fluid is then injected to the VAD by advancing a plunger rod through the syringe containing the medical fluid. After the medical fluid is injected to the VAD, the syringe is disconnected from the VAD and a second pre-filled flushing syringe is used to flush the VAD. Flushing the VAD after administering the medical fluid confirms patency of the catheter and advances any remaining medical fluid in the catheter to the patient ensuring that a full dose of the medical fluid is delivered to the patient.

Connecting or disconnecting a syringe or another device from the VAD introduces portions of the VAD to an unsterile outside environment, which increases a possibility that the VAD will become contaminated and/or will be a source of a bloodstream infection. In order to decrease a risk of bloodstream infection and to ensure VADs are used and maintained correctly, standards of practice require disinfecting and cleaning any fluid ports or connectors of the VAD each time that a new syringe is connected to the VAD. Accordingly, reducing a number of syringes that must be connected to the VAD during a drug delivery procedure simplifies drug administration by reducing a number of times that portions of the VAD must be disinfected and cleaned. Reducing the number of syringes or other fluid delivery devices used during a procedure also reduces opportunities for portions of the VAD to become contaminated, thereby reducing the risk for bloodstream infections. The syringes and methods of the present disclosure provide simplified drug administration procedures by reducing the number of syringes used during such procedures, thereby reducing a risk of contamination and bloodstream infection.

### SUMMARY OF THE INVENTION

According to an aspect of the disclosure, a multi-barrel syringe for sequential expulsion of at least a first fluid followed by a second fluid includes an outer barrel configured to contain the first fluid and an inner barrel slideably inserted in the outer barrel configured to contain the second fluid. The outer barrel includes an open proximal end, a distal end comprising a fluid port, a sidewall extending between the proximal end and the distal end, and at least one protrusion extending into an interior of the outer barrel from an inner surface of the outer barrel. The inner barrel includes an open proximal end, a closed distal end, and a sidewall extending between the proximal end and the distal end. Moving the inner barrel through the outer barrel brings a pierceable portion of the inner barrel into contact with the at least one protrusion of the outer barrel to pierce the inner barrel thereby allowing the second fluid to flow from the inner barrel to the outer barrel. The syringe further includes a plunger at least partially inserted in the inner barrel for moving the inner barrel through the outer barrel and for moving through the inner barrel to expel the second fluid from the inner barrel through the pierceable portion of the inner barrel.

According to another aspect of the disclosure, a pre-filled flushing syringe includes any of the previously described syringes of the present disclosure, with the inner barrel at least partially inserted in the outer barrel and the plunger at least partially inserted in the inner barrel. The pre-filled syringe further includes a predetermined volume of the second fluid in the inner barrel sealed in the inner barrel by the plunger.

According to another aspect of the present disclosure, a method for sequential expulsion of fluids from one of the previously described syringes includes applying distally directed pressure to the plunger, thereby causing the inner barrel to move through the outer barrel to expel the first fluid from the outer barrel through the fluid port. The method further includes, once the pierceable portion of the inner barrel is pierced by the at least one protrusion of the outer barrel, continuing to apply distally directed pressure to the plunger to move the plunger through the inner barrel, thereby expelling the second fluid from the inner barrel to the outer barrel and through the fluid port.

Non-limiting illustrative examples of embodiments of the present disclosure will now be described in the following numbered clauses.

Clause 1: A multi-barrel syringe for sequential expulsion of at least a first fluid followed by a second fluid, the syringe comprising: an outer barrel configured to contain the first fluid comprising an open proximal end, a distal end comprising a fluid port, a sidewall extending between the proximal end and the distal end, and at least one protrusion extending into an interior of the outer barrel from an inner surface of the outer barrel; an inner barrel configured to contain the second fluid slideably inserted in the outer barrel comprising an open proximal end, a closed distal end, and a sidewall extending between the proximal end and the distal end, wherein moving the inner barrel through the outer barrel brings a pierceable portion of the inner barrel into contact with the at least one protrusion of the outer barrel to pierce the inner barrel thereby allowing the second fluid to flow from the inner barrel to the outer barrel; and a plunger at least partially inserted in the inner barrel for moving the inner barrel through the outer barrel and for moving through the inner barrel to expel the second fluid from the inner barrel through the pierceable portion of the inner barrel.

Clause 2: The syringe of clause 1, wherein applying pressure to the plunger moves the inner barrel through the outer barrel when the pierceable portion of the inner barrel is intact.

Clause 3: The syringe of clause 1 or clause 2, wherein, when the pierceable portion of the inner barrel is intact, applying proximal pressure to the plunger moves the inner barrel proximally through the outer barrel to aspirate the first fluid into the outer barrel through the fluid port.

Clause 4: The syringe of any of clauses 1-3, wherein, when the pierceable portion of the inner barrel is intact, applying distal pressure to the plunger moves the inner barrel distally through the outer barrel to expel the first fluid from the outer barrel through the fluid port.

Clause 5: The syringe of any of clauses 1-4, wherein, after the pierceable portion of the inner barrel is pierced by the at least one protrusion, applying distal pressure to the plunger moves the plunger through the inner barrel.

Clause 6: The syringe of any of clauses 1-5, wherein the inner barrel and the outer barrel comprise at least one of polyester, polycarbonate, polypropylene, polyethylene, polyethylene terephthalate, or acrylonitrile butadiene styrene.

Clause 7: The syringe of any of clauses 1-6, wherein the inner barrel comprises a finger flange extending radially from the open proximal end of the inner barrel, and the outer barrel comprises a finger flange extending radially outward from the outer barrel.

Clause 8: The syringe of any of clauses 1-7, wherein the distal end of the inner barrel comprises an annular flange configured to contact an inner surface of the outer barrel.

Clause 9: The syringe of any of clauses 1-8, wherein the inner barrel comprises a stopper mounted to the distal end of the inner barrel sized to seal against an inner surface of the sidewall of the outer barrel.

Clause 10: The syringe of clause 9, wherein the stopper comprises a full stopper which covers the distal end of the inner barrel and a portion of the sidewall of the inner barrel.

Clause 11: The syringe of clause 9 or clause 10, wherein the stopper comprises an annular partial stopper which partially covers the distal end of the inner barrel.

Clause 12: The syringe of any of clauses 1-11, wherein the pierceable portion of the inner barrel comprises a groove in the closed distal end of the inner barrel.

Clause 13: The syringe of clause 12, wherein a thickness of the groove is less than a thickness of other portions of the closed distal end of the inner barrel.

Clause 14: The syringe of clause 12 or clause 13, wherein the groove comprises an annular groove positioned to be contacted by multiple protrusions extending inwardly from the distal end of the outer barrel.

Clause 15: The syringe of any of clauses 1-14, wherein the plunger comprises a stopper slideably positioned in the inner barrel and a plunger rod connected to and extending proximally from the stopper.

Clause 16: The syringe of clause 15, wherein the stopper comprises a thermoplastic elastomer, such as isoprene.

Clause 17: The syringe of clause 15 or clause 16, wherein the plunger rod comprises a press plate at a proximal end of the plunger rod.

Clause 18: The syringe of any of clauses 15-17, wherein the stopper comprises a frustoconical distal surface and at least one annular rib configured to seal against an inner surface of the sidewall of the inner barrel.

Clause 19: The syringe of any of clauses 1-18, wherein the at least one protrusion comprises at least one of a spike, needle, ridge, sharp, point, or post.

Clause 20: The syringe of any of clauses 1-19, wherein the at least one protrusion is integral with other portions of the outer barrel.

Clause 21: The syringe of clause 20, wherein the outer barrel, including the at least one protrusion, is an integrally molded part.

Clause 22: The syringe of any of clauses 1-21, wherein the at least one protrusion extends inwardly from an inner surface of the distal end of the outer barrel.

Clause 23: The syringe of clause 22, wherein the outer barrel comprises a plurality of protrusions extending inwardly from the distal end of the outer barrel.

Clause 24: The syringe of any of clauses 1-23, wherein the at least one protrusion comprises at least one channel for fluid flow from the inner barrel to the outer barrel upon piercing of the pierceable portion of the inner barrel.

Clause 25: The syringe of clause 24, wherein the at least one channel comprises at least one of a groove, notch, opening, slot, cutout, cavity, or lumen at least partially extending through the at least one protrusion.

Clause 26: The syringe of any of clauses 1-25, further comprising a removable cap engaged to the outer barrel and/or the inner barrel for preventing the inner barrel from being fully inserted into the outer barrel while the cap is engaged.

Clause 27: The syringe of clause 26, wherein the removable cap, when engaged to the inner barrel and/or the outer barrel, prevents the pierceable portion of the inner barrel from contacting and being pierced by the at least one protrusion of the outer barrel.

Clause 28: The syringe of clause 26 or clause 27, wherein the removable cap is removably engaged to a finger flange extending radially outwardly from the proximal end of the outer barrel.

Clause 29: A pre-filled flushing syringe, comprising: the syringe of any of clauses 1-28, with the inner barrel at least partially inserted in the outer barrel and the plunger at least partially inserted in the inner barrel; and a predetermined volume of the second fluid in the inner barrel sealed in the inner barrel by the plunger.

Clause 30: The pre-filled syringe of clause 29, wherein the second fluid is a saline flush solution.

Clause 31: The pre-filled syringe of clause 29 or clause 30, further comprising a removable cap engage to the outer barrel and/or the inner barrel for preventing the inner barrel from being fully inserted into the outer barrel while the cap is engaged.

Clause 32: A method for sequential expulsion of fluids from the syringe of any of clauses 1-28, the method comprising: applying distally directed pressure to the plunger, thereby causing the inner barrel to move through the outer barrel to expel the first fluid from the outer barrel through the fluid port; and once the pierceable portion of the inner barrel is pierced by the at least one protrusion of the outer barrel, continuing to apply distally directed pressure to the plunger to move the plunger through the inner barrel, thereby expelling the second fluid from the inner barrel to the outer barrel and through the fluid port.

Clause 33: The method of clause 32, further comprising initially applying proximally directed pressure to the plunger, thereby causing the inner barrel to move proximally through the outer barrel to aspirate the first fluid into the outer barrel.

Clause 34: The method of clause 33, wherein aspirating the first fluid further comprises attaching a needle to the fluid port of a fluid container, inserting the needle into the fluid container, and drawing the first fluid from the fluid container to the outer barrel.

Clause 35: The method of any of clauses 32-34, wherein the syringe is provided partially pre-filled containing a predetermined volume of the second fluid in the inner barrel.

Clause 36: The method of clauses 32-35, further comprising attaching at least one patient line to the fluid port of the syringe prior to expelling the first fluid from the outer barrel through the fluid port.

Clause 37: The method of clauses 32-36, wherein the second fluid comprises a flush solution, such as saline solution and/or heparin lock flush solution, and the first fluid comprises a therapeutic agent.

### BRIEF DESCRIPTION OF THE DRAWINGS

FIG. 1A is a perspective cross-sectional view of a multi-barrel syringe for sequential delivery of a first fluid followed by a second fluid in an initial partially filled state, according to an aspect of the present disclosure.

FIG. 1B is a cross-sectional view of the multi-barrel syringe of FIG. 1A in a filled state.

FIG. 1C is an exploded view of the multi-barrel syringe of FIG. 1A.

FIG. 2A is a cross-sectional view of the multi-barrel syringe of FIG. 1A in the partially filled state.

FIG. 2B is a cross-sectional view of the multi-barrel syringe of FIG. 1A in the filled state.

FIG. 2C is a cross-sectional view of the multi-barrel syringe of FIG. 1A in use to expel fluids from the syringe.

FIG. 2D is a cross-sectional view of the multi-barrel syringe of FIG. 1A in a final or after use state.

FIG. 3A is an enlarged view of a distal portion of an outer barrel of the multi-barrel syringe of FIG. 1A showing protrusions extending from a distal surface of the barrel, according to an aspect of the present disclosure.

FIGS. 3B and 3C are additional views of a distal portion of the multi-barrel syringe of FIG. 1A showing the protrusions of the outer barrel piercing the inner barrel, according to an aspect of the present disclosure.

FIGS. 4A and 4B are cross-sectional views of portions of additional exemplary multi-barrel syringes including stoppers connected to inner barrels of the syringes, according to aspects of the present disclosure.

FIG. 5 is a flow chart showing steps for using a multi-barrel syringe for aspiration and expulsion of fluids from the syringe, according to an aspect of the disclosure.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. As used herein, the term “proximal” refers to a portion or end of a device, such as a syringe or catheter, which is grasped, manipulated, or used by a practitioner or another user. The term “distal” refers to an end or portion of the device that is farthest away from the portion of the device that is grasped, manipulated, or used by the practitioner. For example, the “proximal end” of a catheter or IV line refers to the end including a fluid port that is connected to a fluid container, such as an IV bag or syringe. The “distal end” of the catheter or IV line refers to the end that is connected to the patient. However, it is to be understood that the invention may assume alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as limiting.

With reference to the figures, the present disclosure is directed to multi-barrel syringes 10, 210 that expel a first fluid F1 followed by a second fluid F2 from chambers or barrels of the syringe 10, 210 through a fluid port or nozzle of the syringe 10, 210 to, for example, a vascular access device. The first fluid F1 can be a medical fluid, which, as used herein, can refer to a medication, a total parenteral nutrient (TPN) liquid, or another therapeutic agent used for treatment of chronic or acute conditions, as are known in the art. Exemplary therapeutic agents can include, for example, drugs, chemicals, or biological or biochemical substances that, when delivered in a therapeutically effective amount to the patient, achieve a desired therapeutic effect. The second fluid F2 can be a flush solution, such as saline and/or a heparin lock flush solution. An example of a saline flush solution is 0.9% sodium chloride USP for injection. An example of a heparin lock flush solution is 0.9% sodium chloride with 100 USP units of heparin sodium per mL or 10 USP units of heparin sodium per mL. Other flush solutions, as are known in the art, may also be used with the syringes 10, 210 of the present disclosure. The syringes 10, 210 can also be used for sequential delivery of a first medication or therapeutic agent followed by a different second medication or therapeutic agent.

The syringes 10, 210 of the present disclosure allow a practitioner, such as a medical technician, nurse, physician assistant, physician, or other trained or untrained clinicians or medical caregivers, to administer, for example, a medication followed by a flush solution without needing to change syringes between delivery of the first fluid and the second fluid. Beneficially, the sequential delivery of the medication followed by the flush solution occurs automatically through advancement of, for example, a plunger rod of the syringes 10, 210. As used herein, “advancement” of the plunger rod means that the practitioner is able to push the plunger rod in a distal direction, through a barrel of the syringe 10, 210, as a single stroke to expel the first fluid F1 followed by the second fluid F2 from the syringe 10, 210. The practitioner does not need to, for example, disconnect a syringe or another device from the VAD between delivery of the first fluid and the second fluid. Further, using the syringes 10, 210 of the present disclosure, the practitioner does not need to change a direction of movement of the plunger rod or press another component or mechanism of the syringe 10, 210 in order to perform the sequential delivery of the first fluid and the second fluid.

In some examples, the syringes 10, 210 are provided as a partially pre-filled syringe, where a chamber or barrel of the syringe 10, 210 is filled with a flush solution during manufacturing. The partially pre-filled syringe 10, 210 can include caps, clips, retainers, and/or other packaging to hold the plunger rod in place and to ensure that the flush solution does not leak from the partially pre-filled syringe 10, 210 at unexpected times, such as during transport.

The syringes 10, 210 of the present disclosure are also configured to allow the practitioner to aspirate a medical fluid into the syringe 10, 210 prior to fluid delivery to the patient. For example, the practitioner can insert a nozzle or needle of the syringe 10, 210 into a vial containing the medical fluid and then aspirate the medical fluid into a fluid chamber or barrel of the syringe 10, 210 by moving the plunger rod of the syringe 10, 210 in a proximal direction. After the medical fluid is aspirated into the fluid chamber or barrel of the syringe 10, 210, sequential delivery of the first fluid F1 followed by the second fluid F2 can occur by connecting the syringe 10, 210 to a fluid port of a VAD and then moving the plunger rod of the syringe 10, 210 in the distal direction, thereby expelling the first fluid followed by the second fluid from the syringe 10, 210 to the VAD.

By eliminating the need to clean or disinfect portions of the VAD between delivery of the first fluid and the second fluid, the syringes 10, 210 of the present disclosure simplify the fluid administration procedure, providing substantial time savings compared to conventional fluid administration practices. The syringes 10, 210 of the present disclosure also reduce infection risk and allow for flushing of the VAD immediately following administration of the medical fluid, which may prevent drug occlusion in the VAD. Further, the syringes 10, 210 of the present disclosure have zero dead space, meaning that all medical fluid contained in a first chamber of the syringe 10, 210 is flushed from the syringe 10, 210 as the second fluid (e.g., the flush solution) moves through the syringe 10, 210 to the VAD.

### Multi-Barrel Syringe for Sequential Drug Delivery

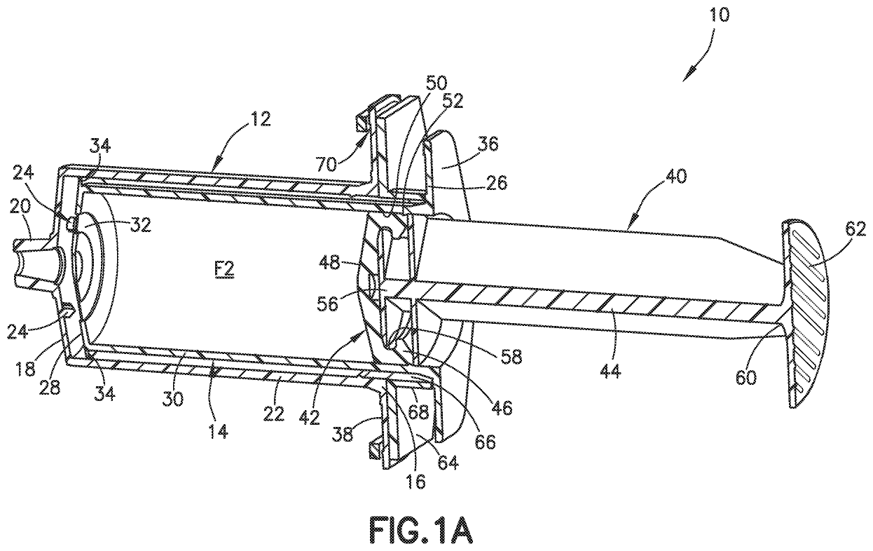

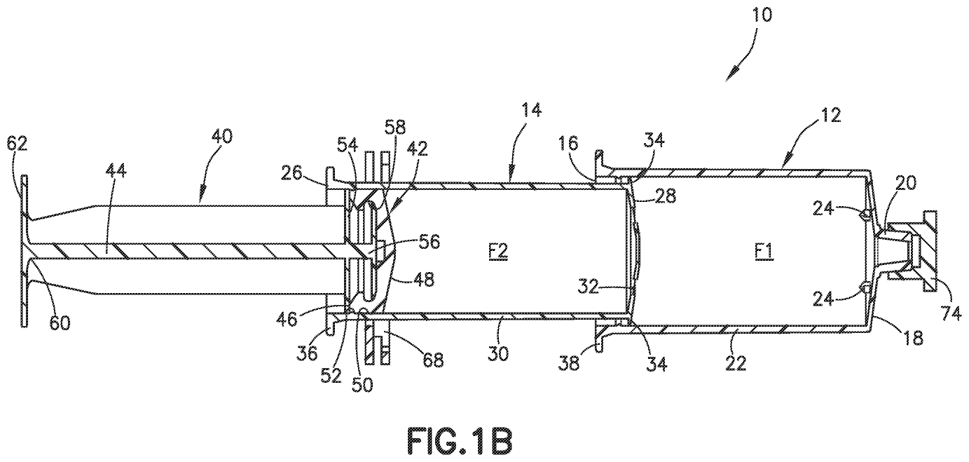

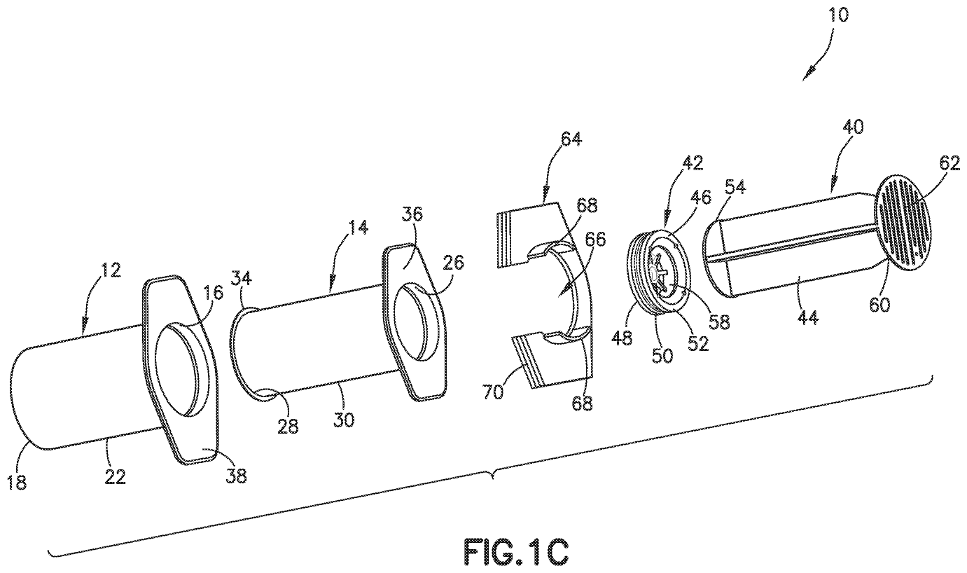

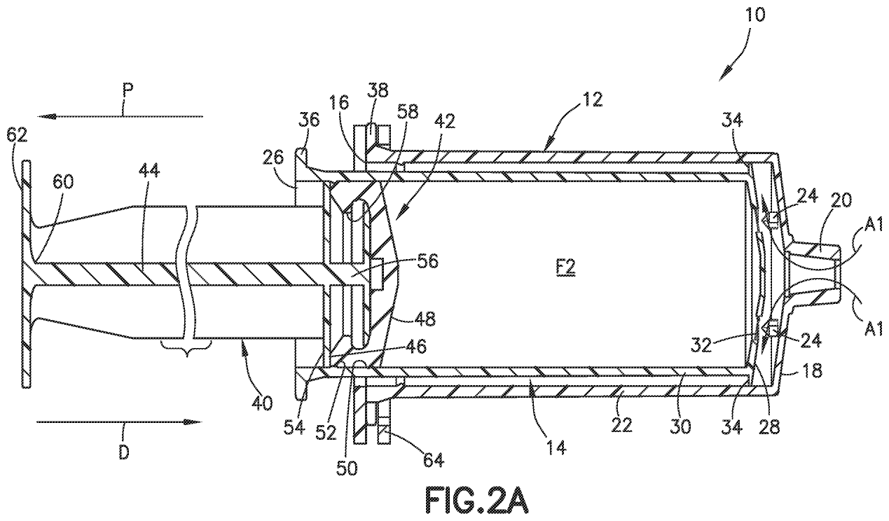

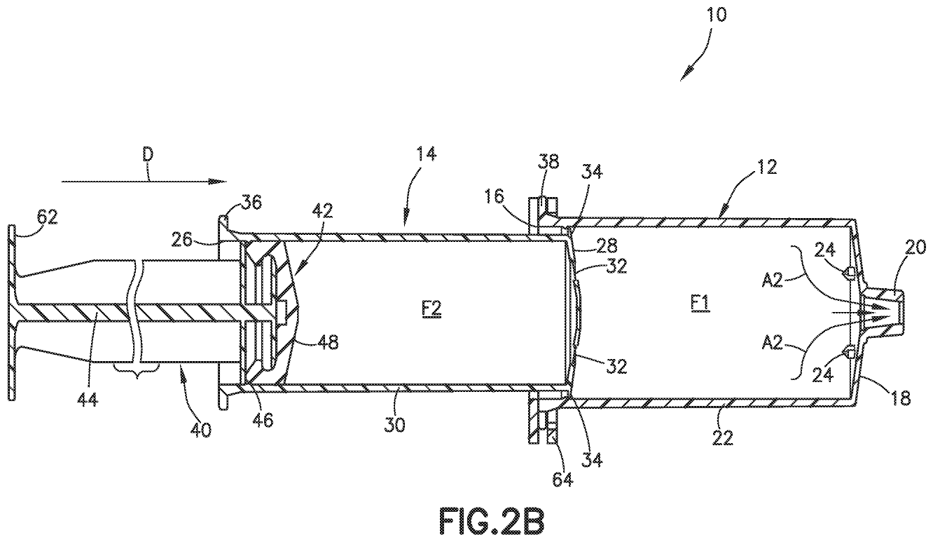

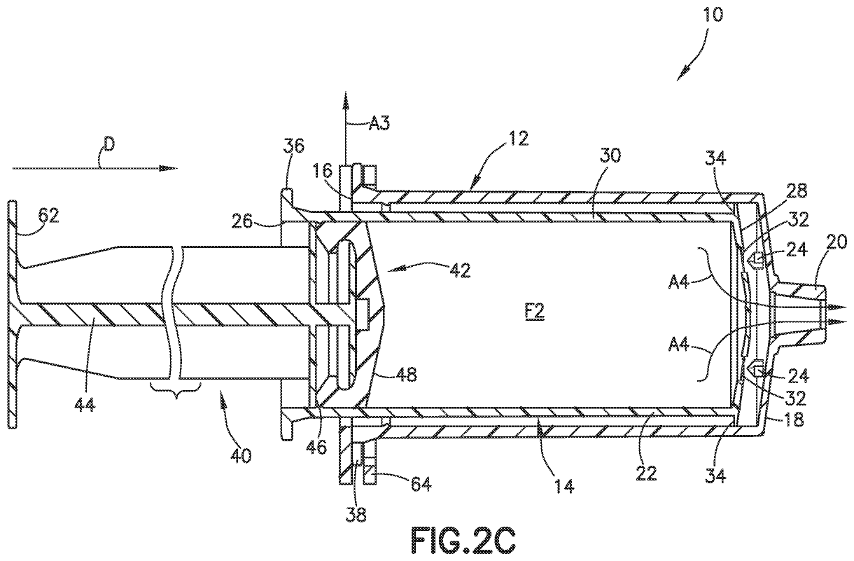

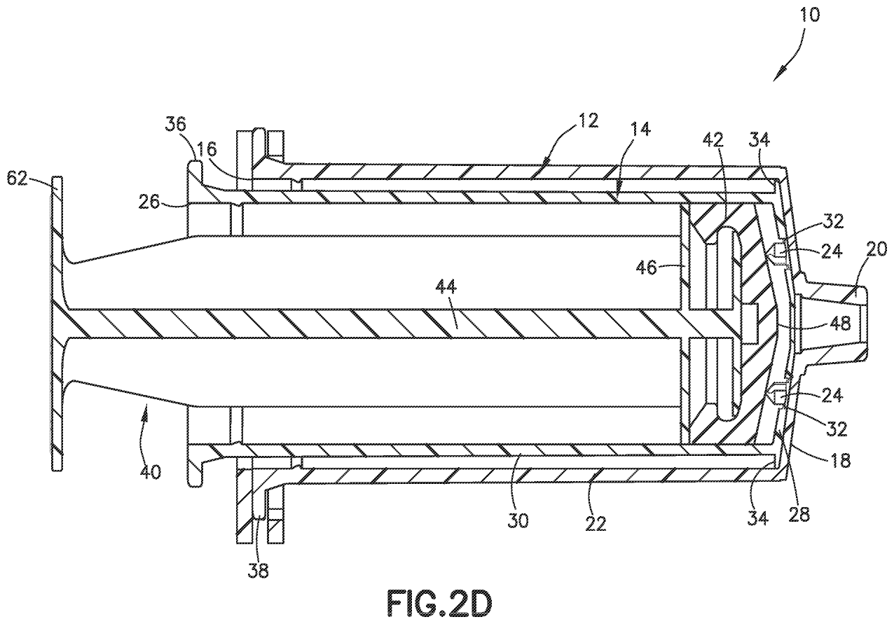

FIGS. 1A-2D illustrate an example of a multi-barrel syringe 10 for sequential expulsion of at least a first fluid F1 followed by a second fluid F2. As previously described, the first fluid F1 can be a medical fluid, such as a drug or another therapeutic agent intended for delivery to a patient through a VAD, such as a catheter or IV line. The second fluid F2 can be a flush solution, such as saline solution and/or an anticoagulant, such as heparin. The type of flush solution and amount of flush solution contained in the syringe 10 may vary depending, for example, on the specific type of catheter or IV line being used. In some examples, the syringe 10 contains or is configured to contain between about 1 mL and 20 mL of the second fluid F2 or, preferably, between about 5 mL and about 10 ml of the second fluid F2.

The syringe 10 comprises an outer barrel 12 configured to contain the first fluid F1 and an inner barrel 14 configured to contain the second fluid F2 slideably inserted in the outer barrel 12. The outer barrel 12 can include an open proximal end 16, a distal end 18 comprising a fluid port 20, and a sidewall 22 extending between the proximal end 16 and the distal end 18. The fluid port 20 of the outer barrel 12 can be a connector, such as a luer connector, threaded connector, or snap connector, configured to be connected to a needle for accessing, for example, an interior of a medical vial containing a medical fluid. The fluid port 20 of the outer barrel 12 can also be configured to be connected directly or indirectly to a fluid port, valve, or another terminal access portion of a VAD. For example, a common type of fluid port of a VAD is a pierceable septum or pre-slit septum made of rubber or another elastomeric material, which permits insertion of a sharp or blunt needle cannula in order to infuse fluids or to withdraw fluids from a catheter of the VAD. Another common fluid port of a VAD is a valve, which does not require a needle for accessing the VAD. Instead, the valve can be activated by a frusto-conically shaped tip of the syringe barrel to provide fluid communication between the interior of the barrel and the VAD.

The outer barrel 12 also includes one or more protrusions 24 extending into an interior of the outer barrel 12 from an inner surface of the sidewall 22 of the outer barrel 12. As described in further detail herein, the protrusions 24 can be, for example, a spike, needle, ridge, sharp, point, post, or similar protruding member that extends into the interior of the outer barrel 12 for piercing the inner barrel 14. In some examples, the protrusions 24 extends proximally from the inner surface of the distal end 18 of the outer barrel 12. In other examples, one or more of the protrusions 24 may extend radially inwardly from an inner surface of the sidewall 22 of the outer barrel 12.

In some examples, the outer barrel 12 of the syringe 10 can be substantially similar in shape, size, and configuration to barrels of syringes used for administering a flush solution to a VAD, as are known in the art. For example, the outer barrel 12 can be a cylindrical structure formed from a rigid thermoplastic material, such as polyester, polycarbonate, polypropylene, polyethylene, polyethylene terephthalate, acrylonitrile butadiene styrene, or other injection moldable or formable resin materials, as are known in the art. Exemplary barrels for flush syringes are described, for example, in U.S. Patent Appl. Pub. No. 2020/0061297, entitled “Flush Syringe Assembly with Controlled Pulsatile Flushing,” which is incorporated herein by reference in its entirety.

The inner barrel 14 of the syringe 10 can be similar in shape and configuration to the outer barrel 12 but is slightly narrower than the outer barrel 12. In particular, the inner barrel 14 can have an outer diameter that is slightly smaller than an inner diameter of the outer barrel 12, so that the inner barrel 14 can slide through the outer barrel 12. The inner barrel 14 comprises an open proximal end 26, a closed distal end 28, and a sidewall 30 extending between the proximal end 26 and the distal end 28. The inner barrel 14 is configured such that moving the inner barrel 14 through the outer barrel 12 pushes the first fluid F1 in the outer barrel 12 through the fluid port 20 and brings a pierceable portion 32 of the inner barrel 14 into contact with the protrusions 24 of the outer barrel 12 to pierce the inner barrel 14. Once the inner barrel 14 is pierced, the second fluid F2 in the inner barrel 14 flows from the interior of the inner barrel 14 through the pierceable portion 32 to the outer barrel 12.

In order to ensure that the inner barrel 14 moves all or substantially all of the first fluid F1 in the outer barrel 12 towards the fluid port 20, the inner barrel 14 can include an annular flange 34 configured to contact and/or seal against the inner surface of the sidewall 22 of the outer barrel 12. For example, as shown in FIGS. 1A-1C, the inner barrel 14 includes the flange 34, which extends radially outwardly from the distal end 28 of the inner barrel 14. In other examples, an annular flange 34 can be provided extending radially from the sidewall 30 of the inner barrel 14. In some examples, there may be multiple annular flanges 34 extending from the distal end 28 and/or sidewall 30 of the inner barrel 14 in order to increase stability of the inner barrel 14 as it moves through the outer barrel 12 and/or to enhance a seal between the inner barrel 14 and the inner surface of the sidewall 22 of the outer barrel 12.

With continued reference to FIGS. 1A-2D, the barrels 12, 14 can include grips, flanges, supports, and similar structures in order to make the syringe 10 easier to hold and manipulate for the practitioner. For example, the inner barrel 14 can comprise a finger flange 36 extending radially outward from the open proximal end 26 of the inner barrel 14. Similarly, the outer barrel 12 comprises a finger flange 38 extending radially outward from the outer barrel 12. The practitioner can grasp the finger flanges 36, 38 between, for example, the ring finger and middle finger, in order to aspirate fluid into the syringe 10 or expel fluid from the syringe 10.

The syringe 10 further comprises a plunger 40 that is slideably inserted in the inner barrel 14. The plunger 40 comprises a stopper 42 slideably positioned in the inner barrel 14 and a plunger rod 44 connected to and extending proximally from the stopper 42. As previously described, the stopper 42 and the plunger rod 44 are configured to move the inner barrel 14 through the outer barrel 12 to expel the first fluid F1 from the outer barrel 12 through the fluid port 20. The plunger rod 44 and stopper 42 are also configured to move through the inner barrel 14 to expel the second fluid F2 from the interior of the inner barrel 14 to the outer barrel 12 through the pierceable portion 32 of the inner barrel 14. More specifically, as described in further detail herein in connection with FIGS. 2A-2D, the inner barrel 14 and plunger 40 can be arranged such that applying pressure to the plunger rod 44 moves the inner barrel 14 through the outer barrel 12 when the pierceable portion 32 of the inner barrel 14 is intact. In particular, when the pierceable portion 32 of the inner barrel 14 is intact, applying proximal pressure to the plunger rod 44 moves the inner barrel 14 proximally through the outer barrel 12 to aspirate the first fluid F1 into the outer barrel 12 through the fluid port 20. In a similar manner, when the pierceable portion 32 of the inner barrel 14 is intact, applying distal pressure to the plunger rod 44 moves the inner barrel 14 distally through the outer barrel 12 to expel the first fluid F1 from the outer barrel 12 through the fluid port 20. After the pierceable portion 32 of the inner barrel 14 is pierced by the protrusions 24, applying distal pressure to the plunger rod 44 moves the plunger rod 44 and the stopper 42 through the inner barrel 14.

The stopper 42 can include many features of conventional syringe stoppers or plungers, as are known in the art. For example, the stopper 42 can be formed from a thermoplastic elastomer material, such as polypropylene or polyethylene, as well as from synthetic or natural rubber (e.g., isoprene). The stopper 42 can include a proximal surface 46 or proximal end, a distal surface 48 or end, and an outer peripheral surface 50 extending between the proximal surface 46 and the distal surface 48. In some examples, the distal surface 48 or distal end of the stopper 42 can be conical or frusto-conical to assist in expelling the second fluid F2 from the inner barrel 14 through the pierceable portion 32 of the inner barrel 14. Alternatively, the distal surface 48 or distal end of the stopper 42 can be substantially flat. The stopper 42 can further include one or more annular ribs 52 protruding from the outer peripheral surface 50. The annular ribs 52 are configured to seal against the inner surface of the sidewall 30 of the inner barrel 14, ensuring that the inner barrel 14 moves with the plunger rod 44 when the pierceable portion 32 of the inner barrel 14 is intact and that the second fluid F2 moves through the inner barrel 14 in an expected manner after piercing the pierceable portion 32 of the inner barrel 14. In some examples, the stopper 42 can include multiple annular ribs 52 in order to improve stability and to prevent the stopper 42 from tilting, shifting, or otherwise deforming as it moves through the inner barrel 14.

The plunger rod 44 is connected to and extends proximally from the stopper 42. The plunger rod 44 can be, for example, an injection molded part formed from a rigid thermoplastic material, such as polyester, polycarbonate, polypropylene, polyethylene, polyethylene terephthalate, or another thermoplastic material, as are known in the art. The plunger rod 44 includes a distal end 54 engaged to the stopper 42. For example, the distal end 54 of the plunger rod 44 can include a connector 56 that is inserted into a corresponding cavity 58 or slot on the proximal surface 46 of the stopper 42. The plunger rod 44 also includes a proximal end 60 protruding proximally from the proximal end 26 of the inner barrel 14. The proximal end 60 of the plunger rod 44 can include a thumb press plate 62 for manipulating the plunger rod 44 to move the stopper 42 and/or the inner barrel 14. The plunger rod 44 can have a variety of cross-sectional shapes and configurations within the scope of the present disclosure. For example, the plunger rod 44 can have a generally cross shaped cross-section. In other examples, the cross-section of the plunger rod 44 can be an I-beam shape, hollow circle, hollow square, hollow rectangle, or L-shaped.

As shown most clearly in FIGS. 1A and 1C, the syringe 10 can also include a removable cap 64 that is engaged to the outer barrel 12 or the inner barrel 14 for preventing the inner barrel 14 from being fully inserted into the outer barrel 12 until the cap 64 is removed. In particular, the cap 64 can be configured to maintain separation between the pierceable portion 32 of the inner barrel 14 and the protrusions 24 of the outer barrel 12 until the cap 64 is removed. Therefore, the removable cap 64, when engaged to the inner barrel 14 and/or the outer barrel 12, prevents the pierceable portion 32 of the inner barrel 14 from contacting and being pierced by the protrusions 24 of the outer barrel 12. As shown in FIG. 1C, the removable cap 64 can be a c-shaped structure including a central opening 66 sized to fit around the outer barrel 12 or the inner barrel 14. The cap 64 also includes vertical walls 68 or tabs that extend vertically for contacting a portion of the inner barrel 14 or the outer barrel 12 to maintain the spacing between the inner barrel 14 and the outer barrel 12. The cap 64 can also include a slot 70 or opening that is sized to fit over the finger flange 36, 38 of either the inner barrel 14 or the outer barrel 12. For example, as shown in FIG. 1A, the removable cap 64 is inserted over the finger flange 38 of the outer barrel 12 and is positioned such that the vertical walls 68 or tabs contact the finger flange 36 of the inner barrel 14 preventing the inner barrel 14 from being fully inserted into the outer barrel 12.

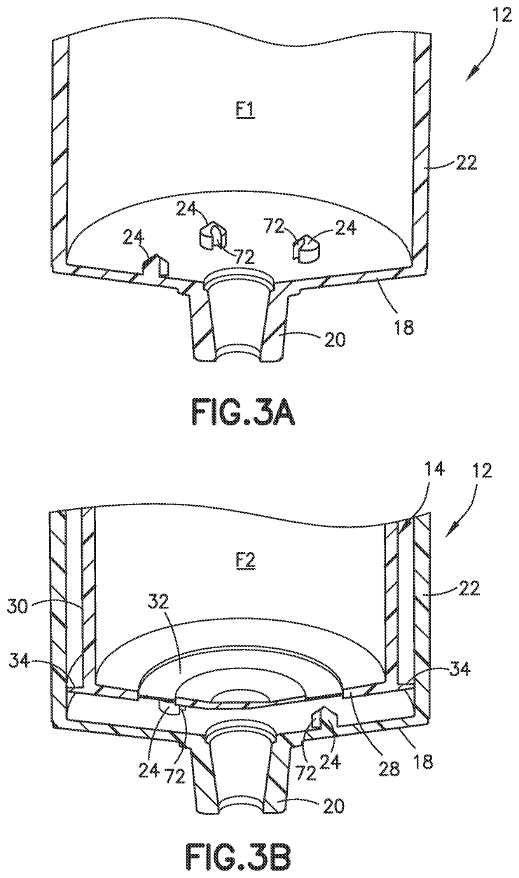

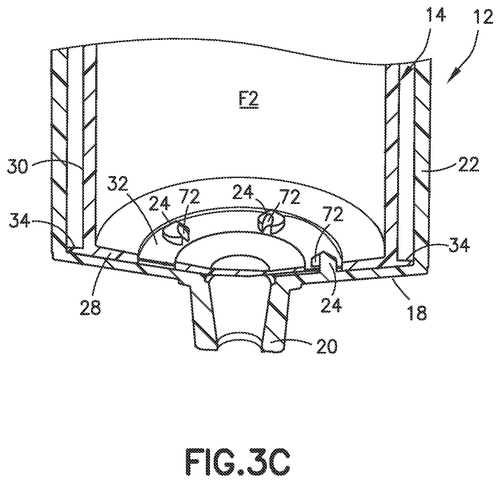

FIGS. 3A-3C show enlarged views of distal portions of the syringe 10 focusing on the protrusions 24 of the outer barrel 12 and the pierceable portion 32 of the inner barrel 14. As previously described, the inner barrel 14 includes a closed distal end 28. As shown in FIGS. 3B and 3C, the closed distal end 28 is substantially flat or may include a slight taper, sloping towards a center of the inner barrel 14. Also as shown in FIGS. 3B and 3C, the inner barrel 14 includes the flange 34 that extends radially outwardly from the closed distal end 28 of the inner barrel 14 contacting the inner surface of the sidewall 22 of the outer barrel 12. As previously described, the inner barrel 14 also includes the pierceable portion 32 which, as shown in FIGS. 3B and 3C, can be positioned on the closed distal end 28 of the inner barrel 14. In some examples, the pierceable portion 32 is a groove on the closed distal end 28 of the inner barrel 14 that is thinner than other portions of the closed distal end 28. The groove can be an annular or circle-shaped groove positioned to be contacted by multiple protrusions 24 extending inwardly from the distal end 18 of the outer barrel 12.

As shown in FIGS. 3A-3C, the protrusions 24 of the outer barrel 12 extend proximally from the distal end 18 of the outer barrel 12. For example, as shown in FIG. 3A, the outer barrel 12 includes multiple points or spikes extending proximally from an inner surface of the distal end 18 of the outer barrel 12. The multiple protrusions 24 are positioned around or encircle the fluid port 20 of the outer barrel 12. The protrusions 24, such as the point or spikes, can be integral with other portions of the outer barrel 12. For example, the outer barrel 12, including the protrusion, can be an integrally molded part formed, for example, by a single injection molding process. In some examples, the protrusions 24 include channels 72, such as groove, notch, opening, slot, cutout, cavity, or lumen, at least partially extending through the protrusions 24. The channels 72 are configured to allow fluid from the inner barrel 14 to flow to the outer barrel 12 upon piercing of the pierceable portion 32 of the inner barrel 14. In particular, as previously described and as shown in FIGS. 3A-3C, the distal end 28 of the inner barrel 14 is initially spaced apart from the distal end 18 of the outer barrel 12. The inner barrel 14 is moved distally through the outer barrel 12 such that the protrusions 24 of the outer barrel 12 contact and pierce the pierceable portion 32 (e.g., the annular groove) at the distal end 28 of the inner barrel 14, as shown in FIG. 3B. After the inner barrel 14 is pierced, as shown in FIG. 3C, fluid can flow through the channels 72 of the protrusions 24 to pass from the inner barrel 14 to the outer barrel 12. Once in the outer barrel 12, the fluid can be expelled from the syringe 10 through the fluid port 20 of the outer barrel 12.

### Method of Expelling Fluid From a Multi-Chamber Syringe

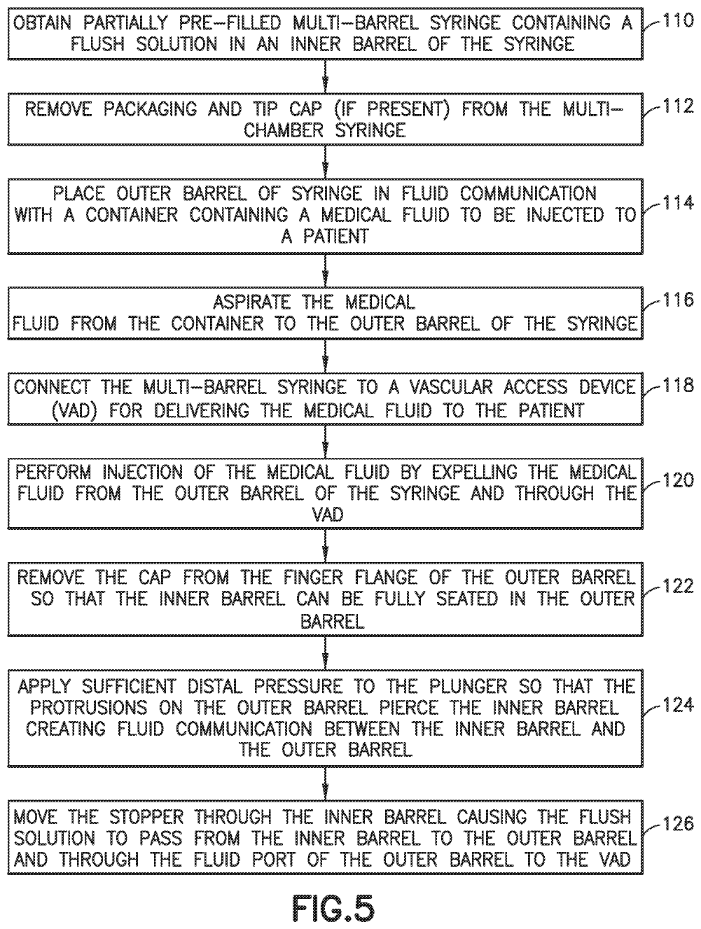

As previously described, the syringe 10 is used for sequential expulsion of fluids, such as expulsion of a medical fluid followed by expulsion of a flush solution from the syringe 10 to the VAD. A flow chart illustrating steps for a sequential fluid expulsion process using the syringe 10 of the present disclosure is shown in FIG. 5.

As shown in FIG. 5, at step 110, a practitioner initially obtains a partially filled syringe 10, in which the inner barrel 14 of the syringe 10 is filled with a predetermined volume of a second fluid F2, such as the flush solution. For example, the partially filled syringe 10 can contain about 1 mL to about 20 mL or, preferably, about 5 mL to 10 mL of the flush solution. The syringe 10 in this initial or partially-filled state is shown in FIG. 2A. At step 112, the practitioner prepares the syringe 10 for use by, for example, removing any packaging, such as a tip cap 74 (shown in FIG. 1B) from the syringe 10. The practitioner may also move the plunger rod 44 in the distal direction, shown by arrow D in FIG. 2A to move the inner barrel 14 as far into the outer barrel 12 as possible without having the inner barrel 14 contact the protrusions 24 extending from the outer barrel 12. In particular, as previously described, the removable cap 64, which is attached to the finger flange 38 of the outer barrel 12 in FIG. 2A, prevents the inner barrel 14 from contacting the protrusions 24. At step 114, the practitioner places the outer barrel 12 in fluid communication with an interior of a container containing the medical fluid to be delivered to the patient. For example, the practitioner may attach a needle (not shown) to the fluid port 20 at the distal end 18 of the outer barrel 12 and insert the needle into the container, such as a medical vial, containing the medical fluid to be injected to the patient.

At step 116, the practitioner aspirates the medical fluid (e.g., the first fluid F1) into the outer barrel 12. For example, the practitioner may grasp the outer barrel 12 and plunger rod 44 (e.g., with two fingers contacting the finger flange 38 of the outer barrel 12 and the thumb contacting the press plate 62 of the plunger rod 44) and move the plunger rod 44 in the proximal direction, shown by arrow P in FIG. 2A, which moves the inner barrel 14 in the proximal direction, thereby aspirating or drawing fluid from an interior of the container into an interior of the outer barrel 12, as shown by arrows A1 in FIG. 2A. The syringe 10 is shown in its fully filled position in FIG. 2B with the first fluid F1 in the interior of the outer barrel 12 and the second fluid F2 in the interior of the inner barrel 14.

Once a dose of the medical fluid (first fluid F1) to be delivered to the patient is drawn into the outer barrel 12, at step 118, the practitioner removes the needle from the fluid port 20 of the outer barrel 12 and connects the fluid port 20 to the VAD. For example, the practitioner may insert a nozzle of the outer barrel 12 into a corresponding port or valve of the VAD, thereby establishing fluid communication between the outer barrel 12 and a lumen of the VAD. At step 120, once the syringe 10 is appropriately connected to the VAD, the practitioner grasps the plunger rod 44 (e.g., by placing a thumb of the press plate 62 and fingers on the finger flange 38 of the outer barrel 12) and pushes the plunger rod 44 in the distal direction (shown by arrow D in FIG. 2B), which causes the inner barrel 14 to move distally through the outer barrel 12. Distal movement of the inner barrel 14 causes the first fluid F1 (e.g., the medical fluid) in the outer barrel 12 to be expelled to the VAD through the fluid port 20 of the outer barrel 12, as shown by arrow A2 in FIG. 2B. The practitioner continues to move the plunger rod 44 in the distal direction until the inner barrel 14 is pressed against the removable cap 64 engaged to the finger flange 38 of the outer barrel 12 indicating that most or all of the first fluid F1 has been expelled from the outer barrel 12. The syringe 10 is shown in this position with the inner barrel 14 contacting the cap 64 in FIG. 2C.

At step 122, the practitioner removes the cap 64 from the finger flange 38 of the outer barrel 12 by, for example, sliding the cap 64 away from the finger flange 38, as shown by arrow A3 in FIG. 2C. Once the cap 64 is removed, the inner barrel 14 is free to move farther into the outer barrel 12. With the inner barrel 14 free to move to its distal-most position within the outer barrel 12, at step 124, the practitioner applies distal pressure to the plunger rod 44 (shown by arrow D in FIG. 2C), which causes the pierceable portion 32 of the inner barrel 14 to contact and to be pierced by the protrusions 24 of the outer barrel 12.

Once the inner barrel 14 is pierced, at step 126, the practitioner continues to push the plunger rod 44 in the distal direction (shown by arrow D), which moves the stopper 42 through the inner barrel 14. Movement of the stopper 42 through the inner barrel 14 causes the second fluid F2, such as the flush solution, in the inner barrel 14 to pass through the channels 72 in the protrusions 24 to the outer barrel 12. The second fluid F2 then passes through the outer barrel 12 and is expelled from the outer barrel 12 to the VAD through the fluid port 20, as shown by arrows A4 in FIG. 2C. The syringe 10 is shown in an end-of-use or final position in FIG. 2D, with the pierced inner barrel 14 fully seated against the inner surface of the distal end 18 of the outer barrel 12 and both the first fluid F1 and the second fluid F2 expelled from the syringe 10.

### Multi-Barrel Syringes Including Stoppers Mounted to the Inner Barrel

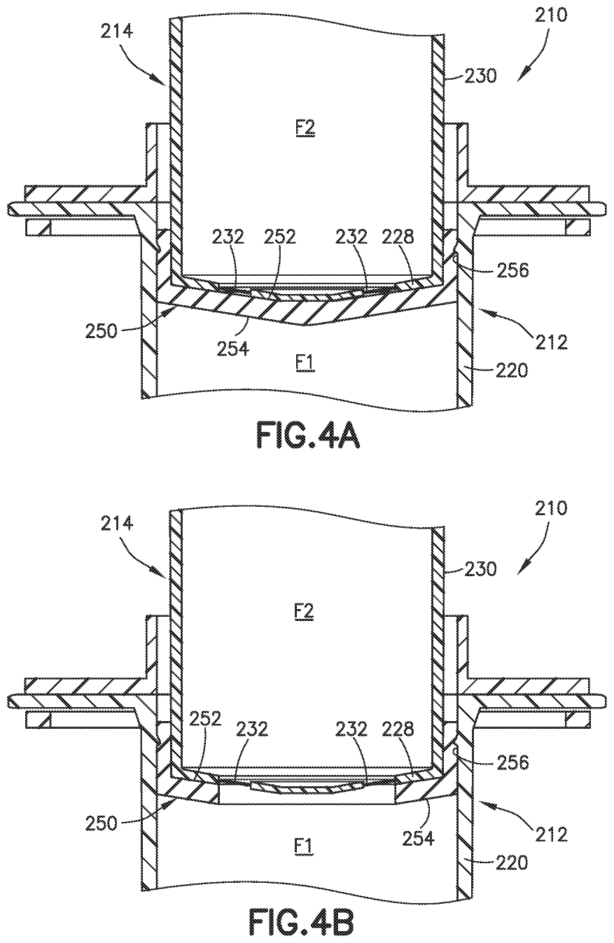

FIGS. 4A and 4B illustrate additional examples of a multi-barrel syringe 210 for sequential delivery of a first fluid F1, such as a medical fluid, followed by a second fluid F2, such as a flush solution. The syringe 210 includes many of the components and features of the syringe 10 shown in FIGS. 1A-3C. In particular, the syringe 210 comprises an outer barrel 212, an inner barrel 214 inserted in the outer barrel 212, and a plunger (not shown in FIGS. 4A and 4B) at least partially inserted in the inner barrel 214. The syringe 210 operates in the same manner as the syringe 10 shown in FIGS. 1A-3C. In particular, a practitioner can grasp and pull or push the plunger to move the inner barrel 214 through the outer barrel 212 while a pierceable portion 232 of the inner barrel 214 is intact. Once the pierceable portion 232 of the inner barrel 214 is pierced by protrusions of the outer barrel 212, applying distally directed pressure to the plunger moves the plunger through the inner barrel 214 to expel the second fluid F2 from the inner barrel 214 into the outer barrel 212 through the pierceable portion 232.

The syringes 210 of FIGS. 4A and 4B differ from previous examples in that the flange on the distal end of the inner barrel shown in FIGS. 1A-3C is replaced by a seal or stopper 250 positioned over a closed distal end 228 of the inner barrel 214. The seal or stopper 250 can include many features of conventional syringe stoppers or plungers, as are known in the art. For example, the seal or stopper 250 can be formed from a thermoplastic elastomer material, such as polypropylene or polyethylene, as well as from synthetic or natural rubber (e.g., isoprene). The seal or stopper 250 can include a proximal surface 252 or proximal end in contact with the closed distal end 228 and with an outer surface of a sidewall 230 of the inner barrel 214. The stopper 250 also includes a distal surface 254 or end opposite the proximal surface 252. In some examples, the distal surface 254 or distal end of the stopper 250 can be conical or frusto-conical to assist in expelling the first fluid F1 from the outer barrel 212. Alternatively, the distal surface 254 or distal end of the stopper 250 can be substantially flat. The stopper 250 can further include an annular sealing surface 256 configured to seal against an inner surface of a sidewall 220 of the outer barrel 212, ensuring that the first fluid F1 moves through the outer barrel 212 in an expected manner.

In some examples, as shown in FIG. 4A, the stopper 250 is a full stopper which covers the distal end 228 of the inner barrel 214. The sealing surface 256 of the stopper 250 extends around a periphery of the closed distal end 228 of the inner barrel 214 and contacts the outer surface of the sidewall 230. As shown in FIG. 4A, the stopper 250 covers the pierceable portion 232 of the inner barrel 214. Accordingly, the protrusions (not shown in FIGS. 4A and 4B) of the outer barrel 212 must be modified from the protrusions shown in FIGS. 1A-3C. In particular, when used with the full stopper 250, the protrusions must be sharp enough to pierce the elastomeric material of the stopper 250. Also, the protrusions must be long enough to protrude through the stopper 250 and pierceable portion 232 of the inner barrel 214 into an interior of the inner barrel 214.

In some examples, as shown in FIG. 4B, the stopper can be an annular or partial stopper 250. The annular or partial stopper 250 partially covers the distal end 228 of the inner barrel 214 and extends about the periphery of the distal end 228 to contact portions of the outer surface of the sidewall 230 of the inner barrel 214. In particular, with the partial or annular stopper 250, the pierceable portion 232 of the inner barrel 214 is not covered by the stopper 250. Accordingly, the protrusions of the outer barrel 212 do not need to pierce the elastomeric material of the stopper 250, as occurs with the full stopper 250 shown in FIG. 4A. However, with the partial or annular stopper 250 of FIG. 4B, the protrusions of the outer barrel 212 must still be long enough to protrude through the pierceable portion 232 of the inner barrel 214 accounting for the thickness of the stopper 250.

While examples of the multi-barrel syringes and methods of the present disclosure are shown in the accompanying figures and described hereinabove in detail, other examples will be apparent to, and readily made by, those skilled in the art without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are to be embraced within their scope.

The invention claimed is:

1. A multi-barrel syringe for sequential expulsion of at least a first fluid followed by a second fluid, the syringe comprising:
an outer barrel configured to contain the first fluid comprising an open proximal end, a distal end comprising a fluid port, a sidewall extending between the proximal end and the distal end, and at least one protrusion extending into an interior of the outer barrel from an inner surface of the outer barrel;
an inner barrel configured to contain the second fluid slideably inserted in the outer barrel comprising an open proximal end, a closed distal end, and a sidewall extending between the proximal end and the distal end, wherein moving the inner barrel through the outer barrel brings a pierceable portion of the inner barrel into contact with the at least one protrusion of the outer barrel to pierce the inner barrel, thereby allowing the second fluid to flow from the inner barrel to the outer barrel; and
a plunger at least partially inserted in the inner barrel for moving the inner barrel through the outer barrel and for moving through the inner barrel to expel the second fluid from the inner barrel through the pierceable portion of the inner barrel.

2. The syringe of claim 1, wherein applying pressure to the plunger moves the inner barrel through the outer barrel when the pierceable portion of the inner barrel is intact.

3. The syringe of claim 1, wherein, when the pierceable portion of the inner barrel is intact, applying proximal pressure to the plunger moves the inner barrel proximally through the outer barrel to aspirate the first fluid into the outer barrel through the fluid port.

4. The syringe of claim 1, wherein, when the pierceable portion of the inner barrel is intact, applying distal pressure to the plunger moves the inner barrel distally through the outer barrel to expel the first fluid from the outer barrel through the fluid port.

5. The syringe of claim 1, wherein, after the pierceable portion of the inner barrel is pierced by the at least one protrusion, applying distal pressure to the plunger moves the plunger through the inner barrel.

6. The syringe of claim 1, wherein the distal end of the inner barrel comprises an annular flange configured to contact an inner surface of the outer barrel.

7. The syringe of claim 1, wherein the inner barrel comprises a stopper mounted to the distal end of the inner barrel sized to seal against an inner surface of the sidewall of the outer barrel.

8. The syringe of claim 7, wherein the stopper comprises a full stopper which covers the distal end of the inner barrel and a portion of the sidewall of the inner barrel.

9. The syringe of claim 7, wherein the stopper comprises an annular partial stopper which partially covers the distal end of the inner barrel.

10. The syringe of claim 1, wherein the pierceable portion of the inner barrel comprises a groove in the closed distal end of the inner barrel.

11. The syringe of claim 1, wherein the plunger comprises a stopper slideably positioned in the inner barrel and a plunger rod connected to and extending proximally from the stopper.

12. The syringe of claim 1, wherein the at least one protrusion comprises at least one of a spike, needle, ridge, sharp, point, or post.

13. The syringe of claim 1, wherein the at least one protrusion is integral with other portions of the outer barrel.

14. The syringe of claim 13, wherein the outer barrel comprises a plurality of protrusions extending inwardly from the distal end of the outer barrel.

15. The syringe of claim 1, where the at least one protrusion comprises at least one channel for fluid flow from the inner barrel to the outer barrel upon piercing of the pierceable portion of the inner barrel.

16. The syringe of claim 15, wherein the at least one channel comprises at least one of a groove, notch, opening, slot, cutout, cavity, or lumen at least partially extending through the at least one protrusion.

17. The syringe of claim 1, further comprising a removable cap engaged to the outer barrel and/or the inner barrel for preventing the inner barrel from being fully inserted into the outer barrel while the cap is engaged.

18. The syringe of claim 17, wherein the removable cap, when engaged to the inner barrel and/or the outer barrel, prevents the pierceable portion of the inner barrel from contacting and being pierced by the at least one protrusion of the outer barrel.

19. A pre-filled flushing syringe, comprising:
the syringe of claim 1, with the inner barrel at least partially inserted in the outer barrel and the plunger at least partially inserted in the inner barrel; and
a predetermined volume of the second fluid in the inner barrel sealed in the inner barrel by the plunger.

20. A method for sequential expulsion of fluids from the syringe of claim 1, the method comprising:
applying distally directed pressure to the plunger, thereby causing the inner barrel to move through the outer barrel to expel the first fluid from the outer barrel through the fluid port; and
once the pierceable portion of the inner barrel is pierced by the at least one protrusion of the outer barrel, continuing to apply distally directed pressure to the plunger to move the plunger through the inner barrel, thereby expelling the second fluid from the inner barrel to the outer barrel and through the fluid port.
